(19) United States
(12) Patent Application Publication
(10) Pub. No.: US 2026/0020748 A1
(43) Pub. Date: Jan. 22, 2026

(54) COLONOSCOPE SHEATH AND METHODS OF USE THEREOF

(71) Applicant: CurtoTech LLC, Northbrook, IL (US)

(72) Inventor: Denise Reynish, Northbrook, IL (US)

(21) Appl. No.: 19/069,990

(22) Filed: Mar. 4, 2025

Related U.S. Application Data
(63) Continuation of application No. 17/219,062, filed on Mar. 31, 2021, now abandoned.
(60) Provisional application No. 63/002,858, filed on Mar. 31, 2020.

(51) Int. Cl. A61B 1/00; A61B 1/31

(52) U.S. Cl. CPC A61B 1/00135; A61B 1/00103; A61B 1/00128; A61B 1/31

(57) ABSTRACT

Disclosed herein is a sheath for reducing looping during insertion of an endoscope during an endoscopic procedure. The sheath may include a lumen operable for receiving a shaft of the endoscope, one or more attachment mechanisms operable to secure the sheath to the endoscope, and a lubricious external surface comprising a lubricant along the entire length of the sheath. The lubricant may be operable to lubricate the movement of the endoscope shaft through a patient and the sheath may cover the entire length of the endoscope shaft prior to being inserted into the patient.

### CROSS-REFERENCE TO RELATED APPLICATIONS

This application claims priority to U.S. Provisional Application No. 63/002,858, filed Mar. 31, 2020, the contents of which are entirely incorporated by reference herein.

### FIELD

The present disclosure relates to an endoscope sheath and methods of use thereof. More specifically, the disclosure relates to a lubricated disposable colonoscope sheath for the reduction or prevention of looping during use and advancement of the colonoscope.

### BACKGROUND

Colonoscopy is a common medical procedure and the chief diagnostic method for colorectal cancer, the second leading cause of cancer-related death. In a colonoscopy, a physician advances a flexible, camera-tipped tube, often also featuring one or more lumens for the passage of water, air, other fluid, or medical devices used in the procedure, through the colon. After the scope is advanced sufficiently into the colon, the physician retracts the scope, slowly, examining the bowel wall for abnormalities.

In order to cut down on procedure time, which incurs costs to the physician, facility, and/or payor, by way of labor, equipment, workspace, and other overhead; as well as risk to the patient via anesthesia, infection, or other means, the provider advances the scope as quickly as possible, since examination is chiefly done upon extraction. This speed is negated by the risk of “looping,” wherein the scope forms an alpha-or n-loop due to the external manipulation, torque, and the flexibility of the bowel wall. These loops, if not resolved, can rupture the bowel wall, which can result in sepsis and other complications for the patient. Though ruptures are rare, time spent resolving loops to prevent them is not.

Therefore, there is a need for an apparatus to prevent entirely or aid in resolution of loops during endoscopic procedures, such as colonoscopies.

### SUMMARY

This disclosure provides a sheath for reducing looping during insertion and advancement of an endoscope. The sheath may include a lumen operable for receiving a shaft of the endoscope; one or more attachment mechanisms operable to secure the sheath to the endoscope; and a lubricious external surface comprising a lubricant along the entire length of the sheath. The lubricant is operable to lubricate the movement of the endoscope shaft through a patient, and the sheath covers the entire length of the endoscope shaft prior to being inserted into the patient. The endoscope may be a colonoscope. The lubricant may be a coating on the lubricious surface or may be embedded in the lubricious surface and is released from the lubricious surface as the sheath is advanced through the patient. The one or more attachment mechanisms may be located on an internal surface of the sheath and/or may be located at the distal end and/or the proximal end of the sheath. The one or more attachment mechanisms may include at least one of a rubber band or rolled segment, a groove, an internal ridge, a variable thickness, and/or a high friction interior surface. The sheath may include an open distal end or a transparent distal end. The sheath may be transparent and/or the lubricious external surface may include one or more distance markings. The sheath may be disposable.

The disclosure further provides a method of reducing looping during an endoscopic procedure of a patient. The method may include covering a length of a shaft of the endoscope with a sheath by inserting the shaft into a lumen of the sheath; attaching the sheath to the shaft of the endoscope using one or more attachment mechanisms; and inserting the covered endoscope shaft into the patient. A lubricious external surface of the sheath may include a lubricant along the entire length of the sheath, such that the lubricant lubricates the movement of the endoscope shaft through the patient. The endoscope may be a colonoscope and the endoscopic procedure may be a colonoscopy. The lubricant may be a coating on the lubricious surface or embedded in the lubricious surface and is released from the lubricious surface as the sheath is advanced through the patient. The one or more attachment mechanisms may be located on an internal surface of the sheath and/or at the distal end and/or the proximal end of the sheath. The one or more attachment mechanisms may include at least one of a rubber band or rolled segment, a groove, an internal ridge, a variable thickness, and/or a high friction interior surface. The sheath may be pre-sterilized and disposable after the procedure. The endoscopic procedure time may be reduced by up to about 20% as compared to when the endoscopic procedure is performed without the sheath. The sheath may reduce friction through the colon by up to about 20% as compared to when the colonoscopy is performed without the sheath. There may be at least about 50% fewer incidents of looping during the endoscopic procedure than when the procedure is performed without the sheath or no incidents of looping during the endoscopic procedure.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered to be limiting of its scope, the principles herein are described and explained with additional specificity and detail through the use of the accompanying drawings in which:

FIG. 1A shows a sheath with a groove and ridges at the distal end and one or more additional grooves/ridges on the internal surface of the sheath along its length.

FIG. 1B is a cross-sectional view of the sheath of FIG. 1A.

FIG. 2A shows a sheath with a lip at the distal end of the sheath to grip the top of the endoscope.

FIG. 2B is a cross-sectional view of the sheath of FIG. 2A.

FIG. 3A shows a sheath with rolled edges at both the proximal and distal ends of the sheath, such that the sheath may be applied to the endoscope shaft using a condom-like application.

FIG. 3B is a cross-sectional view of the sheath of FIG. 3A.

FIG. 4A shows a sheath with a variable thickness, with 1× thickness at and near the distal end, 6× thickness at and near the proximal end, and 3× thickness between.

FIG. 4B is a cross-sectional view of the sheath of FIG. 4A.

FIG. 5 shows a sheath with a narrowed proximal end and rolled edges at the proximal and distal ends of the sheath.

FIG. 6 shows a sheath with a rubber-band connection at the distal end and a clip near the proximal end of the sheath.

The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

As used herein, the terms “comprising,” “having,” and “including” are used in their open, non-limiting sense. The terms “a,” “an,” and “the” are understood to encompass the plural as well as the singular. Thus, the term “a mixture thereof” also relates to “mixtures thereof.”

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

Provided herein is a tubular sheath for reducing and/or preventing looping during insertion of an endoscope. As used herein, “looping” refers to when the endoscope (e.g. colonoscope) stretches and distends the colon in response to advancing the scope forward anywhere the scope encounters a barrier and the endoscope forms an alpha- or n-loop. Once a loop has formed, it must be straightened because these loops, if not resolved, can rupture the bowel wall, which can result in sepsis and other complications for the patient. The sheath may be biosafe, user-friendly, and perform with quality and reliability. In various examples, the sheath may include one or more of the following: a lubricious external surface or other slip promotion/friction reduction, an attachment mechanism, disposable materials, individual packaging, and/or transparent material.

The sheath includes a lubricious outer surface operable to lubricate the movement of the endoscope shaft through a patient. Without being limited to any one theory, the lubricious outer surface of the sheath may prevent or reduce looping during an endoscopic procedure. The lubricious external surface may include a lubricant along the entire length of the sheath such that the entire length of the covered endoscope shaft may move more easily and be less likely to loop than an endoscope shaft without a sheath. This is in contrast to the application of a lubricant directly on a small portion of the endoscope shaft at the start of the procedure, which would only be on a portion of the endoscope shaft and would not be maintained through an entire endoscopic procedure. In some examples, the sheath may be pre-lubricated. The lubricious surface may be a coating of a lubricant or may have an embedded lubricant that is released from the surface. In some examples, a lubricious coating may include an oil, a hydrophilic or hydrophobic lubricant, or hydrophilic and hydrophobic materials that reduce friction. Non-limiting examples of lubricants include seed oil, water-soluble lubricants, petroleum-based lubricants, and silicone-based lubricants. In one example, a hydrophilic or hydrophobic material may be constructed in such a way as to encourage the formation of micro-layers which easily slide past one another. In other examples, the sheath may include a lubricious outer surface that maintains lubrication by releasing lubricant from a bioresorbable material, such as a micro-embedded lubrication that is released upon micro-abrasion of the device. In other examples, the lubricious surface may be directly incorporated on the endoscope surface itself.

The sheath also includes at least one attachment mechanism to secure the sheath to the endoscope or colonoscope. The one or more attachment mechanisms may be located on an internal surface of the sheath or at a proximal end and/or distal end of the sheath. Multiple attachment mechanisms are contemplated, included a rubber-band-like connection, at least one groove or internal ridge designed to couple to a matching ridge or groove on an endoscope, a rolled edge, a clip, a variable thickness such that the sheath is tightly wound around the scope to prevent dislodging, and/or a high friction interior surface. In at least one example, the sheath may be ribbed or textured on the internal surface to help grip the endoscope. In some examples, a different attachment mechanism may be selected for a given endoscope. The attachment mechanism may be formed integral with (e.g. as part of) the sheath or may be a separate component attached to an external surface of the sheath. The sheath and/or attachment mechanisms may be injection molded, dip molded, or cast molded to form the sheath with the attachment mechanisms.

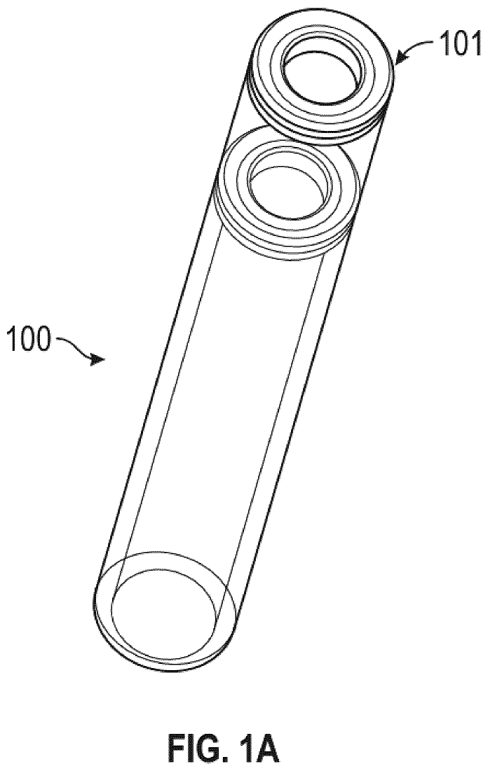

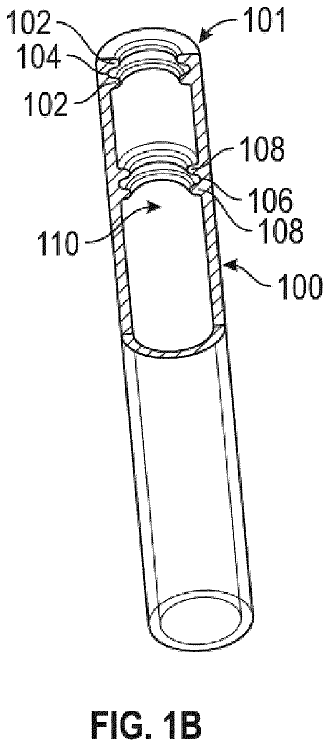

FIGS. 1-6 show various examples of sheaths and attachment mechanisms for the sheath. The figures are not drawn to scale. FIGS. 1A and 1B show a sheath 100 with an attachment mechanism formed by a groove 104 and ridges 102 at the distal end 101 of the sheath 100 that are operable to connect to a slight groove on the top of an endoscope. For example, the sheath 100 may include one groove 104 and two ridges 102, one on either side of the groove 104, essentially forming the groove 104 at the distal end of the sheath 100. The groove 106 and ridges 108 may be circumferential around the inner surface of the sheath 100. The sheath 100 may include one or more additional grooves/ridges on the internal surface of the sheath along its length. In an example, the sheath 100 may include one or more additional grooves 106 and ridges 108 at a location proximal to the groove 104 and ridges 102 at the distal end of the sheath. The sheath 100 further includes a lumen 110 operable for receiving all or part of the endoscope. The groove/ridges may provide varying tightness to secure the sheath to the endoscope shaft. In an example, the sheath may be injection molded to form the attachment mechanism(s).

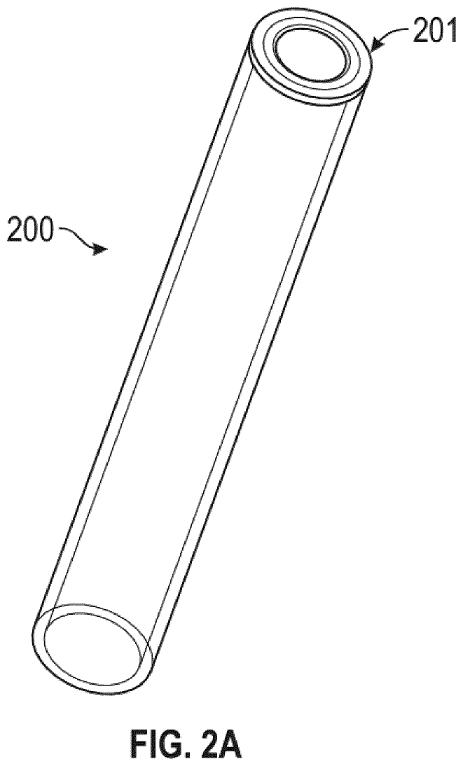

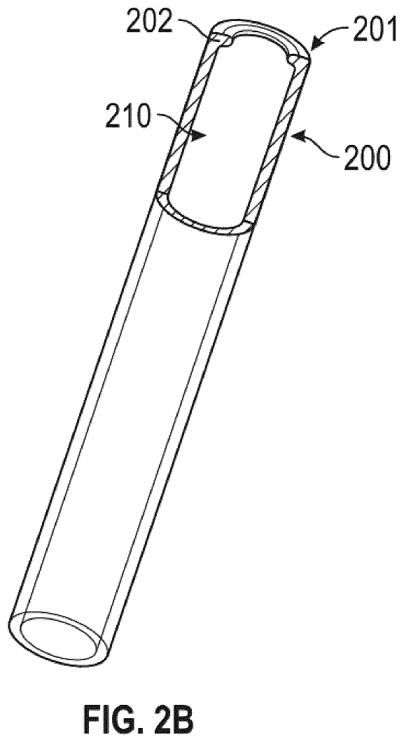

FIGS. 2A and 2B show a sheath 200 with a lip 202 at the distal end 201 of the sheath 200 to grip the top of the endoscope 210 or a groove at the distal end of the endoscope 210. The lip 202 may be circumferential around the inner surface of the sheath 200. The sheath 200 further includes a lumen 210 operable for receiving all or part of the endoscope. In an example, the sheath may be injection molded.

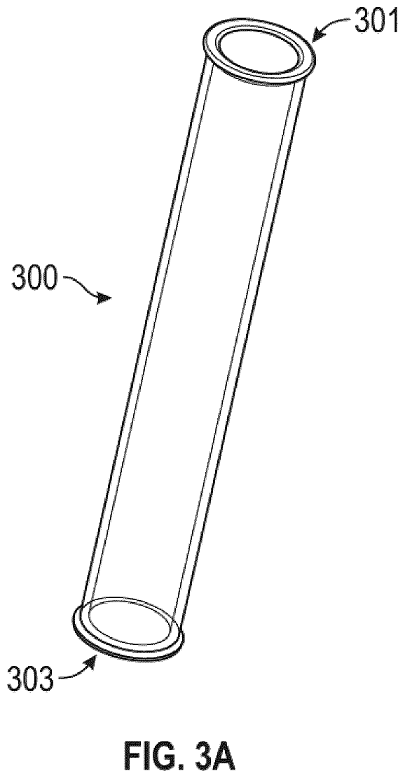

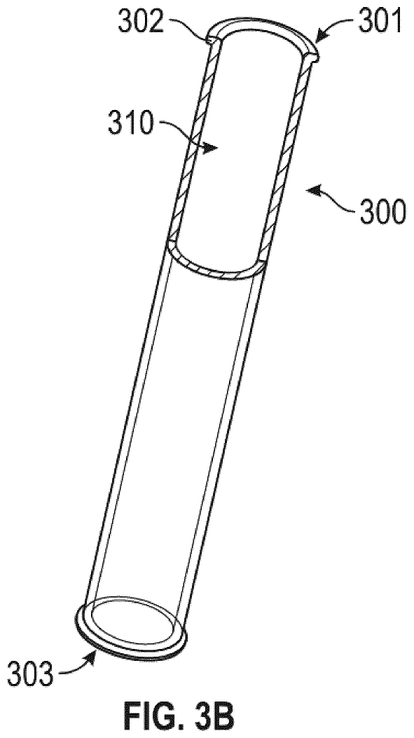

FIGS. 3A and 3B show a sheath 300 with rolled edges 302, 304 at both the proximal 303 and distal 301 ends of the sheath 300. The rolled edges 302, 304 may provide tension on the endoscope to retain the sheath in place. The rolled edges 302, 304 may be circumferential around the distal 301 and proximal 303 ends of the sheath 300. In an example, the sheath 300 may be applied to the endoscope shaft using a condom-like application. The sheath 300 further includes a lumen 310 operable for receiving all or part of the endoscope. In an example, the sheath may be dip molded.

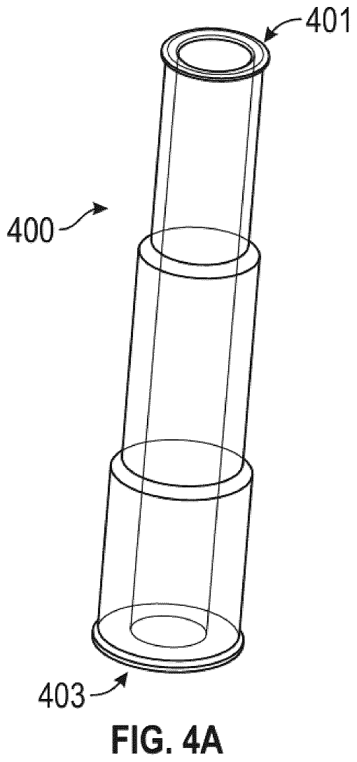

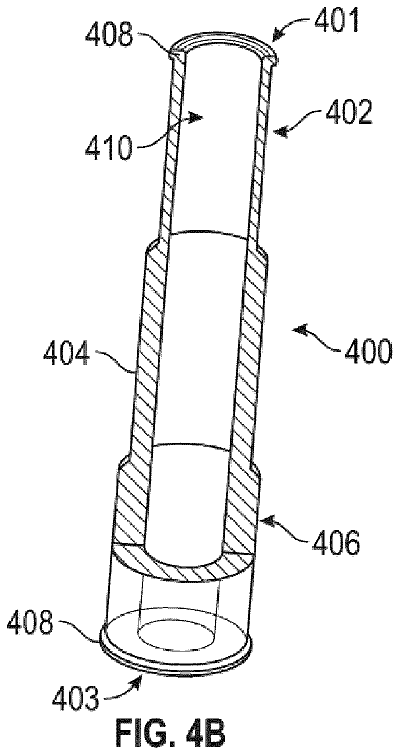

FIGS. 4A and 4B show a sheath 400 with a variable thickness. The sheath 400 may have a first portion 402 with 1× thickness at and near the distal end 401, a second portion 404 with 3× thickness, and a third portion 406 with 6× thickness at and near the proximal end 403. The sheath 400 may also include a rolled edge 408 or ridge at the distal end 401 and/or the proximal end 403. The rolled edges 408 may be circumferential around the distal 401 and/or proximal 403 ends of the sheath 400. The sheath 400 further includes a lumen 410 operable for receiving all or part of the endoscope. In an example, the sheath may be dip molded to form the multiple thicknesses.

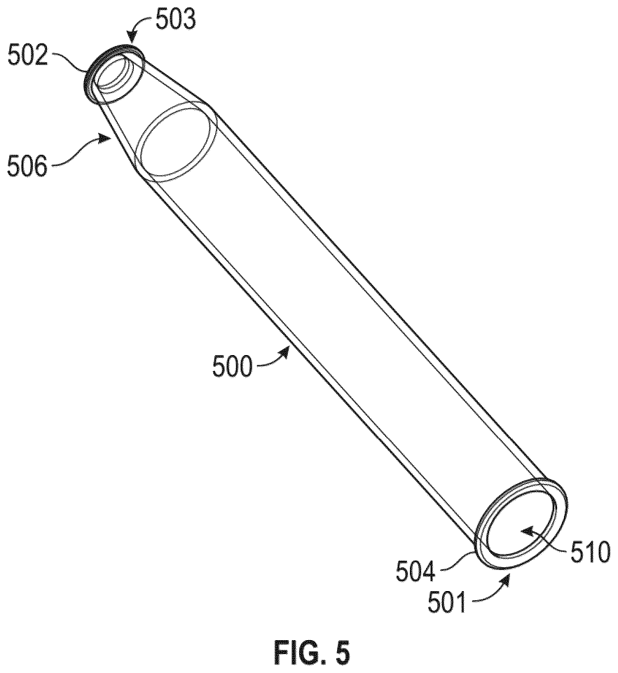

FIG. 5 shows a sheath 500 with a narrowed proximal end 503 and rolled edges 502, 504 at the proximal 503 and distal 501 ends of the sheath 500. The rolled edges 408 may be circumferential around the distal 401 and/or proximal 403 ends of the sheath 400. The sheath may be tapered 506 toward the proximal end 503 such that the diameter of the sheath decreases over a prescribed distance. The sheath 500 further includes a lumen 510 operable for receiving all or part of the endoscope. In this example, the sheath may be dip molded.

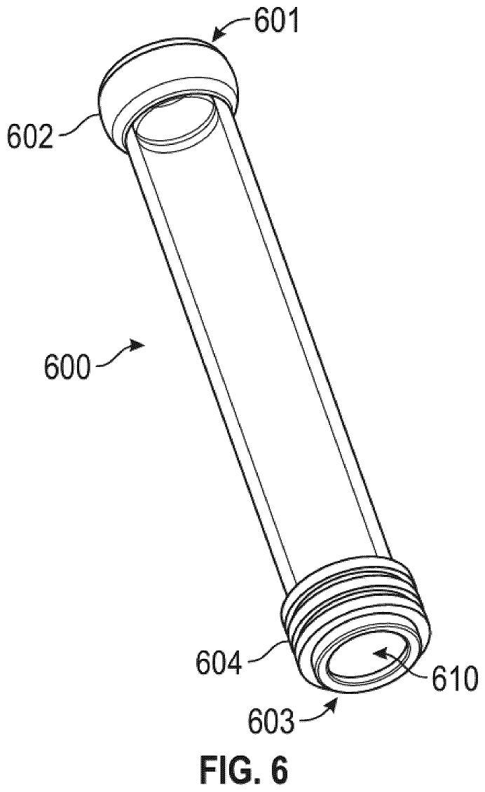

FIG. 6 shows a sheath 600 with a rubber-band connection 602 at the distal end 601 and a clip 604 near the proximal end 603 of the sheath 600. The clip 604 is operable to attach to the outer surface of the sheath 600. The sheath 600 further includes a lumen 610 operable for receiving all or part of the endoscope. In an example, two dip molds may be used to form the sheath, one of the rubber-band connection and one for the complete sheath.

The sheath may have a length sufficient to cover an endoscope's insertable portion, with enough thickness to maintain structural integrity throughout insertion, but not so thick as to add considerable width to the insertable endoscope. In at least one example, the sheath covers the length of an endoscope, such as the entire length of a colonoscope. In various examples, the sheath may have a length of about 40 cm to about 60 cm, about 30 cm to about 50 cm, about 50 cm to about 70 cm, about 40 cm to about 50 cm, about 50 cm to about 60 cm, or any length that a person of ordinary skill in the art would know to be appropriate for a certain colonoscope/other endoscope. In various examples, the sheath may have a thickness of about 0.1 mm to about 5 mm, about 0.5 mm to about 1 mm, about 1 mm to about 2 mm, about 2 mm to about 3 mm, about 4 mm to about 5 mm, or any thickness that would maintain structural integrity of the sheath whole minimizing thickness of the endoscope. In at least one example, the sheath may have a thickness of about 0.5 mm to about 1.5 mm.

The sheath may be made from materials that are bio-safe, qualify for FDA manufacturing standards, and are easily sterilized. The sheath may further be made from inorganic materials to prevent allergic reactions in patients. In some examples, the sheath may be made of a bio-absorbable material or materials that may have lubricant filled sacs inside. For example, the sheath may be made of thermoplastics suitable for injection and dip-molding. In various examples, at least a portion of the sheath may include polyisoprene, isoprene rubber latex, or polyisoprene latex. The sheath may be made through dip molding, injection molding, extrusion, or a combination thereof.

In some examples, the sheath may have a clear/transparent tip (distal end) to allow easy or unimpeded visibility of the endoscope camera. In additional examples, the sheath may be open at the distal end or have a mesh tip that allows the endoscope channels and camera to function properly. In additional examples, the sheath body may be clear/transparent or include external distance markings, such that a physician can identify how much scope has been inserted at any given time.

The sheath may be able to withstand the demands of an endoscopic procedure (i.e. colonoscopy) in a variety of environmental conditions, such as tensile strength, elasticity, tear resistance, heat resistance, pH resistance, and/or rigidity.

The sheath may be pre-sterilized for single-use or may be sterilizable. In some examples, the sheath may be disposable and/or individually packaged to maintain sterility between patients.

The sheath may help to resolve the looping problem that arises during endoscopic procedures, increase colonoscopy success rates, reduce force and pressure along colonic walls during scope advancement, reduce time associated with a colonoscopy procedure, reduce need for excess anesthesia and therefore reduce associated anesthesia costs, reduce patient discomfort post procedure, reduce rates of post procedural presentation back to the ER, result in fewer microperforations/micro-abrasions throughout the procedure, decrease ergonomic strain on medical professionals performing the procedure, increase overall lubrication, reduce risk of bowel perforations, no decrease in scope visibility, not decrease the ability to identify polyps, and/or decrease spasticity of the colon. In various examples, the endoscope sheath may not harm the patient by falling off the endoscope, bunching, rupturing, or negatively impacting the digestive flora.

The sheath may be a user friendly apparatus that fits easily into the current clinician workflow. For example, the sheath may not pose an extra burden to a physician's daily routine and may not have a substantial time increase associated with sheath application on the endoscope. In addition, the sheath may minimize the time of the endoscopic procedure.

A method of reducing looping during insertion of an endoscope into a patient may include covering a length of a shaft of the endoscope with the sheath by inserting the shaft into a lumen of the sheath, attaching the sheath to the shaft of the endoscope using one or more attachment mechanisms, and inserting the covered endoscope shaft into the patient. The lubricious external surface of the sheath includes a lubricant along the entire length of the sheath, such that the lubricant lubricates the movement of the endoscope shaft through the patient.

In some examples, the sheath may be attached to the endoscope before an endoscopic procedure, in its entirety, by a technician. In other examples, the sheath may be attached to the endoscope progressively throughout the endoscopic procedure (e.g. rolling down), in order to prevent destruction of the lubricious coating and maintain quality handling of the scope itself.

In some embodiments, the use of the sheath on an endoscope for an endoscopic procedure, such as a colonoscopy, may reduce the amount of time the procedure would take without the use of the sheath. For example, the use of the sheath may reduce procedure time by up to about 5%, up to about 10%, up to about 15%, up to about 20%, up to about 25%, up to about 30%, up to about 35%, or up to about 40%.

In some embodiments, the use of the sheath on a colonoscope for a colonoscopy may reduce friction through the colon by to about 5%, up to about 10%, up to about 15%, up to about 20%, up to about 25%, up to about 30%, up to about 35%, or up to about 40%. In some examples, the reduction in friction may lead to about 10%, about 20%, about 30%, about 40%, about 50%, about 60%, about 70%, about 80%, about 90%, or up to 100% fewer incidents of looping during the procedure than if the procedure were performed without the sheath.

In some embodiments, the use of the sheath on an endoscope for an endoscopic procedure, such as a colonoscopy, may have a cecal intubation rate of at least 90%, at least 95%, or at least 98%. The cecal intubation rate (CIR) is a standard unit of measure for colonoscopy success. One of the main reasons for incomplete colonoscopies is excessive looping. Because use of the sheath reduces the occurrence of looping, the CIR may increase and the cecal intubation time (CIT) may decrease as compared to a procedure without the use of the sheath. In some embodiments, the CIT may decrease by about 15%, about 20%, or about 25% as compared to the cecal intubation time in a procedure without use of the sheath. That increased CIR and decreased CIT may lead to reduced anesthesia time (risk to patient), reduced procedural anesthesia cost (benefit to facilities), increased patient satisfaction, and decreased risk of having to repeat the colonoscopy procedure.

In an embodiment, the use of the sheath on an endoscope for an endoscopic procedure, such as a colonoscopy, may reduce patient pain as compared to the procedure without the use of the sheath. In another embodiment, the use of the sheath on an endoscope for an endoscopic procedure, such as a colonoscopy, may increase patient satisfaction with the procedure as compared to the procedure without the sheath. In a further embodiment, the use of the sheath on an endoscope for an endoscopic procedure, such as a colonoscopy, may reduce the amount or duration of anesthesia used in the procedure as compared to the amount or duration of anesthesia that would be used without the use of the sheath.

Having described several embodiments, it will be recognized by those skilled in the art that various modifications, alternative constructions, and equivalents may be used without departing from the spirit of the disclosure. Additionally, a number of well-known processes and elements have not been described in order to avoid unnecessarily obscuring the present disclosure. Accordingly, the above description should not be taken as limiting the scope of the disclosure.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A sheath for reducing looping during insertion of an endoscope, the sheath comprising:
a lumen operable for receiving a shaft of the endoscope;
one or more attachment mechanisms operable to secure the sheath to the endoscope; and
a lubricious external surface comprising a lubricant along the entire length of the sheath, wherein the lubricant is operable to lubricate the movement of the endoscope shaft through a patient,
wherein the sheath covers the entire length of the endoscope shaft prior to being inserted into the patient.

2. The sheath of claim 1, wherein the endoscope is a colonoscope.

3. The sheath of claim 1, wherein the lubricant is a coating on the lubricious surface.

4. The sheath of claim 1, wherein the lubricant is embedded in the lubricious surface and is released from the lubricious surface as the sheath is advanced through the patient.

5. The sheath of claim 1, wherein the one or more attachment mechanisms are located on an internal surface of the sheath.

6. The sheath of claim 1, wherein the one or more attachment mechanisms are located at the distal end and/or the proximal end of the sheath.

7. The sheath of claim 1, wherein the one or more attachment mechanisms comprise at least one of a rubber band or rolled segment, a groove, an internal ridge, a variable thickness, and/or a high friction interior surface.

8. The sheath of claim 1, wherein the sheath comprises an open distal end or a transparent distal end.

9. The sheath of claim 1, wherein the lubricious external surface comprises one or more distance markings.

10. The sheath of claim 1, wherein the sheath is disposable.

11. A method of reducing looping during an endoscopic procedure of a patient, the method comprising:
covering a length of a shaft of the endoscope with a sheath by inserting the shaft into a lumen of the sheath;
attaching the sheath to the shaft of the endoscope using one or more attachment mechanisms; and
inserting the covered endoscope shaft into the patient, wherein a lubricious external surface of the sheath comprises a lubricant along the entire length of the sheath, such that the lubricant lubricates the movement of the endoscope shaft through the patient.

12. The method of claim 11, wherein the endoscope is a colonoscope and the endoscopic procedure is a colonoscopy.

13. The method of claim 12, wherein the sheath reduces friction through the colon by up to about 20% as compared to when the colonoscopy is performed without the sheath.

14. The method of claim 11, wherein the lubricant is a coating on the lubricious surface.

15. The method of claim 11, wherein the lubricant is embedded in the lubricious surface and is released from the lubricious surface as the sheath is advanced through the patient.

16. The method of claim 11, wherein the one or more attachment mechanisms are located on an internal surface of the sheath and/or at the distal end and/or the proximal end of the sheath.

17. The method of claim 11, wherein the one or more attachment mechanisms comprise at least one of a rubber band or rolled segment, a groove, an internal ridge, a variable thickness, and/or a high friction interior surface.

18. The method of claim 11, wherein the sheath is pre-sterilized and disposable after the procedure.

19. The method of claim 11, wherein the endoscopic procedure time is reduced by up to about 20% as compared to when the endoscopic procedure is performed without the sheath.

20. The method of claim 11, wherein there are at least about 50% fewer incidents of looping during the endoscopic procedure than when the procedure is performed without the sheath.
